(19) United States
(12) Patent Application Publication
(10) Pub. No.: US 2012/0046452 A1
(43) Pub. Date: Feb. 23, 2012

(54) ANTIBODY-MEDIATED REJECTION INHIBITOR

(75) Inventors: Hideki Ohdan, Hiroshima (JP); Toshimitsu Irei, Hiroshima (JP)

(73) Assignee: HIROSHIMA UNIVERSITY, HIGASHI-HIROSHIMA-SHI, HIROSHIMA (JP)

(21) Appl. No.: 13/318,090

(22) PCT Filed: Apr. 19, 2010
(86) PCT No.: PCT/JP2010/002825

§ 371 Date: Oct. 28, 2011

(30) Foreign Application Priority Data
Apr. 30, 2009 (JP) 2009-110887

(51) Int. Cl. C07K 16/28; G01N 33/53

(52) U.S. Cl. 530/388.15; 530/389.6; 530/388.7; 435/7.92

(57) ABSTRACT

A purpose of the present disclosure to achieve transplantation from ABO-incompatible donors and inhibition of autoimmune disease, for example, without significantly impairing defense mechanisms.

An antibody-mediated rejection inhibitor for impairing signal transduction between NKT-cells and B-cells to inhibit production of antibodies indicates anti-CD1d antibodies.

### TECHNICAL FIELD

The present disclosure relates to antibody-mediated rejection inhibitors.

### BACKGROUND ART

With recent progress in medical technologies, organ transplantations are being generalized for treatment. Nevertheless, organ donors are seriously in short supply. A possible safe transplantation from ABO-incompatible donors can be a solution for the shortage of donors.

Such an organ transplantation from an ABO-incompatible donor, however, has a serious problem of antibody-mediated rejection. Specifically, antigens having blood group A- or B-carbohydrate chains (hereinafter also referred to as blood group carbohydrate chain antigens) are targeted, and antigen-antibody reaction causes organs for transplantation to be out of use.

To prevent the problem described above, immunosuppression treatment prescribing multiple drugs have been proposed. However, these treatments only have uncertain and temporary effects, and are nonspecific to uniformly suppress all the immune functions, resulting in a problem of declines in immune functions.

Under such circumstances, studies have been conducted, focusing on a phenomenon in which lymphocytic cells play important roles in antigen-antibody reaction. For example, based on the finding that anti-HM1.24 antibodies inhibit activation of B-cells and T-cells, an inhibitor of activation of lymphocytes, capable of inhibiting blast formation of T-cells and production of antibodies of B-cells is proposed (see PATENT DOCUMENT 1).

Inventors of the present disclosure also conducted a study on B-cells showing reactivity with the blood group carbohydrate chains described above. Up to the present time, the inventors have confirmed that receptors reactive with blood group carbohydrate chains belong to B-1 cells among B-cells, differentiation of the B-1 cells is inhibited by calcineurin inhibitors, and the calcineurin inhibitors do not affect differentiation of B-2 cells reactive with peptide antigens (corresponding to antigens derived from general foreign enemies such as microbe and virus) (NON-PATENT DOCUMENTS 1-4).

### CITATION LIST

### Patent Document

PATENT DOCUMENT 1: Japanese Patent No. 3552898

### Non-Patent Document

NON-PATENT DOCUMENT 1: Irei T, Ohdan H, Ishiyama K, Tanaka Y, et al., “The persistent elimination of B cells responding to blood group A carbohydrates by synthetic group A carbohydrates and B-1 cell differentiation blockade: novel concept in preventing antibody-mediated rejection in ABO-incompatible transplantation,” Blood, 110 (13): 4567-4575, 2007
NON-PATENT DOCUMENT 2: Ohdan H, Zhou W, Tanaka Y, Irei T, et al., “Evidence of immune tolerance to blood group antigens in a case of ABO-incompatible pediatric liver transplantation,” Am J Transplant, 7 (9): 2190-4, 2007
NON-PATENT DOCUMENT 3: Zhou W, Ohdan H, Tanaka Y, Hara H, et al., “NOD/SCID mice engrafted with human peripheral blood lymphocytes can be a model for investigating B cells responding to blood group A carbohydrate determinant,” Transpl Immunol, 12 (1): 9-18, 2003
NON-PATENT DOCUMENT 4: Ohdan H, Swenson K G, Huw S. Gray K, et al., “Mac-1-Negative B-1b Phenotype of Natural Antibody-Producing Cells, Including Those Responding to Galα1, 3Gal Epitopes inα1, 3-Galactosyltransferase-Deficient Mice,” The Journal of Immunology, 165: 5518-5529, 2000

### SUMMARY OF THE INVENTION

### Technical Problem

Based on the aforementioned results of the study, the inventors are investigating the use of both an anti-CD20 antibody preparation and a calcineurin inhibitor. Specifically, cells which have been already differentiated to B-1 cells are intended to be inhibited using an anti-CD20 antibody preparation showing specific inhibition of B cells, and differentiation of B-1 cells is intended to be inhibited using a calcineurin inhibitor.

The anti-CD20 antibody preparation, however, acts on all the B-cells, and inhibits not only B-1 cells reactive with blood group carbohydrate chains but also B-2 cells reactive with peptide antigens. Accordingly, immune functions decline, resulting in an inevitable increase in infectious diseases.

It is therefore an object of the present disclosure to provide an antibody-mediated rejection inhibitor which is more specific than an anti-CD20 antibody preparation, for example, and can reduce a decline in immune functions.

### Solution to the Problem

As a result of an intensive study focused on natural killer T (NKT) cells, inventors of the present disclosure found that inhibition of signal transduction between NKT-cells and B-cells can lead to advantageous effects.

Specifically, the present disclosure is directed to an antibody-mediated rejection inhibitor for impairing signal transduction between NKT-cells and B-cells to inhibit production of antibodies. The antibody-mediated rejection inhibitor indicates anti-CD1d antibodies. Specifically, the B-cells indicate B-1 cells, and the antibodies to be a target for producing inhibition indicate antibodies against carbohydrate chain antigens. More specifically, the carbohydrate chain antigens indicate blood group carbohydrate chain antigens.

Accordingly, it is possible to inhibit production of at least antibodies against blood group A- or B-carbohydrate chain antigens. Thus, antibody-mediated rejection occurring in, for example, organ transplantation can be effectively inhibited.

On the other hand, the antibodies to be a target for producing inhibition do not include antibodies against peptide antigens. Accordingly, production of antibodies against antigens derived from general foreign enemies is not inhibited, thereby reducing an excessive decline in immune functions.

In addition, if the anti-CD1d antibodies may be monoclonal antibodies, antibody-mediated rejection can be more specifically inhibited. Further, if the anti-CD1d antibodies will be human monoclonal antibodies, successful transplantation from ABO-incompatible donors can be achieved, thus significantly solving the shortage of donors.

The “natural killer T (NKT) cells” are lymphocytic cells having markers of both natural killer (NK) cells and T-cells and receiving attention as cells connecting natural immunity and acquired immunity. NKT cells recognize antigens presented by class I-like MHC molecules, CD1d, with antigen receptors (invariant T cell antigen receptor: iTCR; Vα24TCR in humans and Vα14TCR in mice). As the antigens, a galactosylceramide which is a carbohydrate chain antigen was found. It was generally accepted that NKT-cells produces large amounts of interferon-gamma (IFN-γ) and interleukin-4 (IL-4) when activated, and affect immune systems.

The “B-cells” are lymphocytic cells which differentiate into mature cells in the bone marrow, and antibody forming precursor cells which produce antibodies with predetermined stimulation. As surface markers, CD 19, 20, 21 are provided. The B-cells are classified into general B-cells (B-2 cells) responsible for production of antibodies against protein antigens and B-1 cells responsible for production of antibodies against carbohydrate chain antigens. The “B-1 cells” are mainly present in abdominal cavities, have a surface marker of IgM^{high}CD5⁺, and control production of antibodies against carbohydrate chain antigens independently of T-cells. The “anti-CD1d antibodies” are antibodies against CD1d molecules.

The “carbohydrate chain antigens” means antigenic carbohydrates composing either glycoproteins or glycolipids. Antibody production against carbohydrate chain antigens is assumed to be mainly performed by B-1 cells, and are independent of T-cells. The “blood group carbohydrate chain antigens” are carbohydrate chain antigens found as isoantigens in red blood cells, and are classified into A, B, and O groups. In the brood group A, N-acetylgalactosamine is bonded to O determinants. In the blood group B, β-galactose is bonded to O determinants. The blood group carbohydrate chain antigens are expressed on the vascular endothelium except for red blood cells in organs. In organ transplantation, acute antibody-mediated rejection is caused by natural antibodies against blood group carbohydrate chain antigens and antibodies produced from activated B-cells or plasma cells.

The “monoclonal antibodies” are antibodies against single antigens. One B-cell has a B-cell receptor against a single antigen, is differentiated to a plasma cell after antigen stimulation, and produces immunoglobulin of only one type. Antibodies are produced in xenogenic plasmacytoma, and then are purified, thus obtaining monoclonal antibodies. The “human (chimeric) antibodies” are obtained by replacing constant regions of monoclonal antibodies produced in xenogenic plasmacytoma with constant regions of human immunoglobulin to greatly reduce side effects in administration to humans.

Such an antibody-mediated rejection inhibitor can be obtained by a screening method including the steps of: administering a target component in abdominal cavities of mice to obtain test mice, while administering an antibody component to be inhibited in abdominal cavities of other mice to obtain comparative mice; and immunizing each of the test mice and the comparative mice with red blood cells, thereby measuring production of anti-blood group antibodies.

### Advantages of the Invention

As described above, an antibody-mediated rejection inhibitor according to the present disclosure can provide specific inhibition only of antibody-mediated rejection occurring in, for example, transplantation. This inhibitor enables transplantation from ABO-incompatible donors and inhibition of development of autoimmune diseases.

### BRIEF DESCRIPTION OF THE DRAWINGS

FIG. 1 is a conceptual view for explaining functions in an embodiment of the present disclosure.

FIGS. 2(a) and 2(b) are graphs showing test results in a first example,

FIG. 2(a) shows values before immunization, and FIG. 2(b) shows values obtained two weeks after immunization.

FIGS. 3(a) and 3(b) are graphs showing test results in the first example,

FIG. 3(a) shows values before immunization, and FIG. 3(b) shows values obtained six weeks after immunization.

FIG. 4 is a graph showing test results in a second example.

FIGS. 5(a) and 5(b) are graphs showing test results in a third example, FIG. 5(a) shows values before immunization, and FIG. 5(b) shows values obtained six weeks after immunization.

FIGS. 6(a) and 6(b) are graphs showing test results in the third example, FIG. 6(a) shows values before immunization, and FIG. 6(b) shows values obtained six weeks after immunization.

FIG. 7 is a graph showing test results in a fourth example.

### DESCRIPTION OF EMBODIMENTS

An embodiment of the present disclosure will be described hereinafter with reference to the drawings. The following embodiment is merely a preferred example in nature, and is not intended to limit the scope, applications, and use of the invention.

First, inventors of the present disclosure had an idea that NKT-cells might participate in production of antibodies (also referred to as anti-blood group antibodies) against blood group carbohydrate chain antigens in some way, and the possibility thereof was analyzed. Although detailed analysis will be described in a first example, mice deficient in CD1d molecules (also referred to as knockout mice) and normal mice were immunized with human red blood cells, and then production of anti-blood group antibodies in these mice were compared.

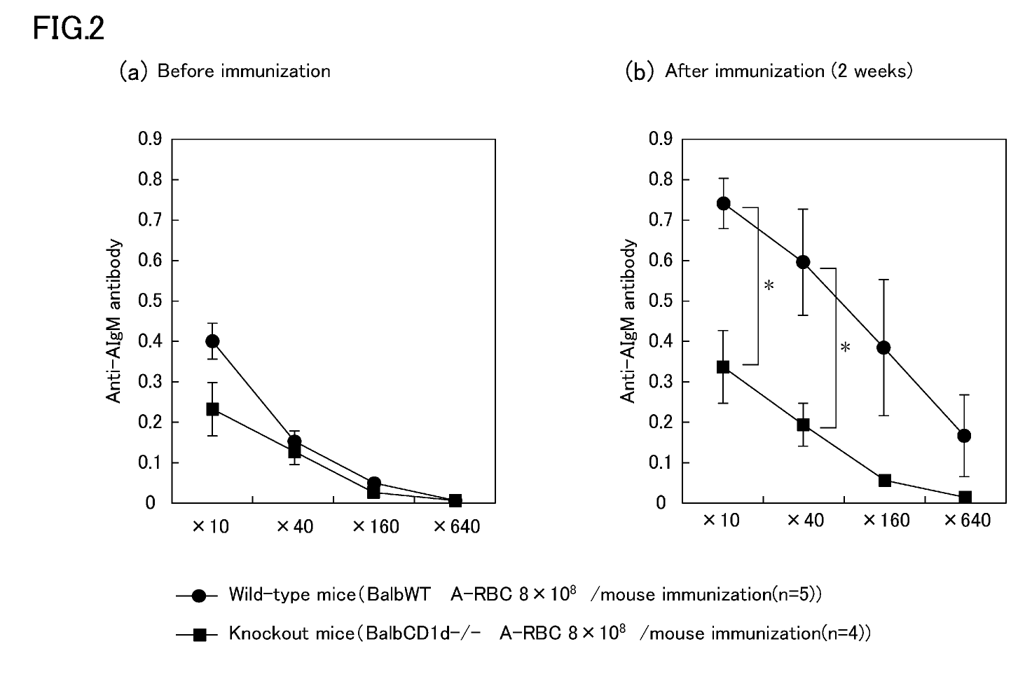

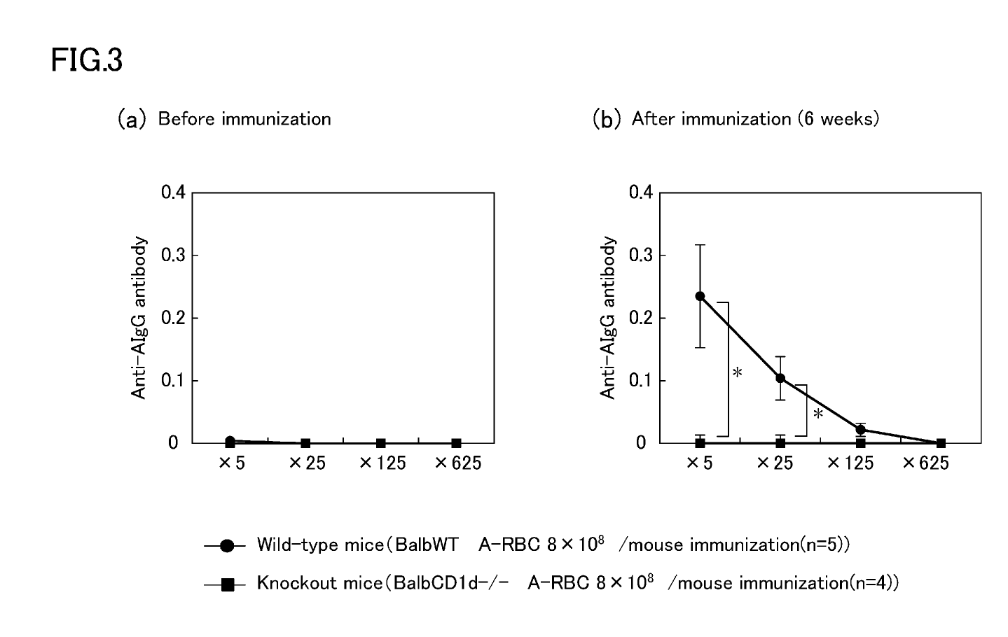

This comparison confirmed that production of anti-blood group antibodies of the immunized knockout mice degrade as compared to the normal mice. In the normal mice, class switching of antibodies, i.e., production of antibodies of another isotype such as IgG, was observed after appearance of immunoglobulin M (IgM), but was not observed in the knockout mice at all (see FIGS. 2(a), 2(b), 3(a), and 3(b)).

Accordingly, it was confirmed that NKT-cells participate in production of anti-blood group antibodies, and are essential for class switching of antibodies.

Next, to confirm whether the above-mentioned participation of NKT-cells in production of anti-blood group antibodies is specific or not, the influence of NKT-cells on production of antibodies against general peptide antigens was analyzed (where detailed description thereof will be given in a second example). Specifically, knockout mice and normal mice were immunized with allogeneic mouse cells, to compare production of anti-alloantibodies.

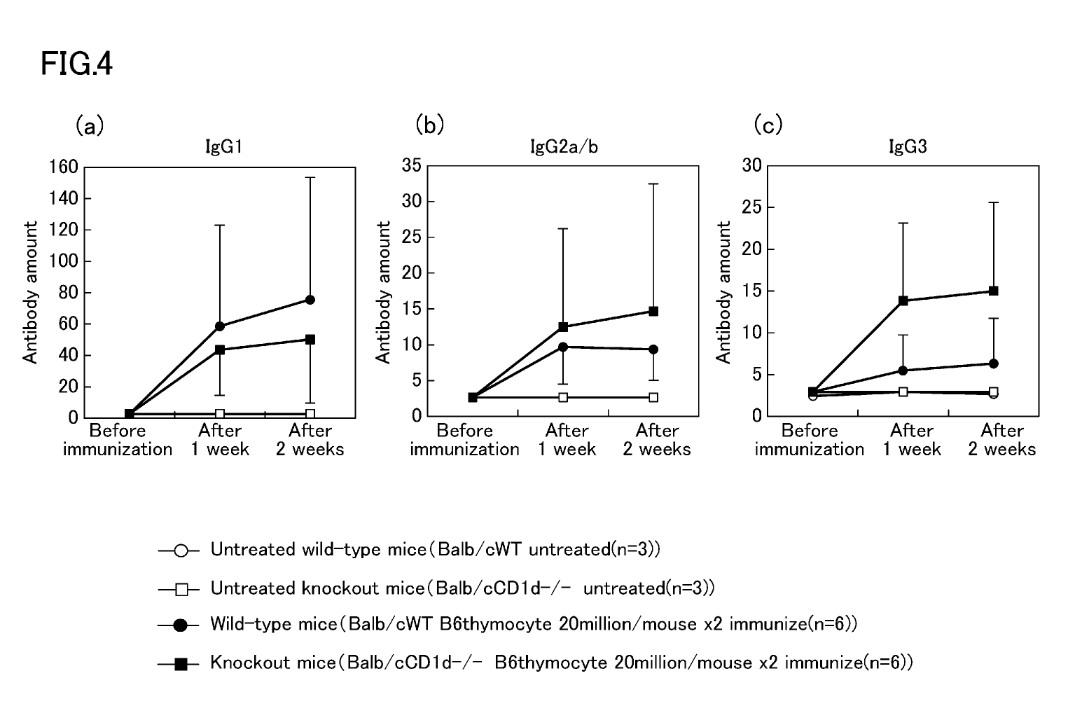

This comparison confirmed that production of anti-alloantibodies and class switching were confirmed after immunization in both of the knockout mice and the normal mice (see FIG. 4).

Accordingly, it was confirmed that the influence of NKT-cells on peptide antigens is small.

The foregoing results suggest that control of activation of NKT-cells can provide specific inhibition of production of anti-blood group antibodies. Thus, the inventors focused on the point that the initial signal transduction between NKT-cells and B-cells is performed by presenting antigens through CD1d molecules, and hypothesized that impairment of signal transduction between NKT-cells and B-cells by anti-CD1d antibodies can inhibit production of anti-blood group antibodies.

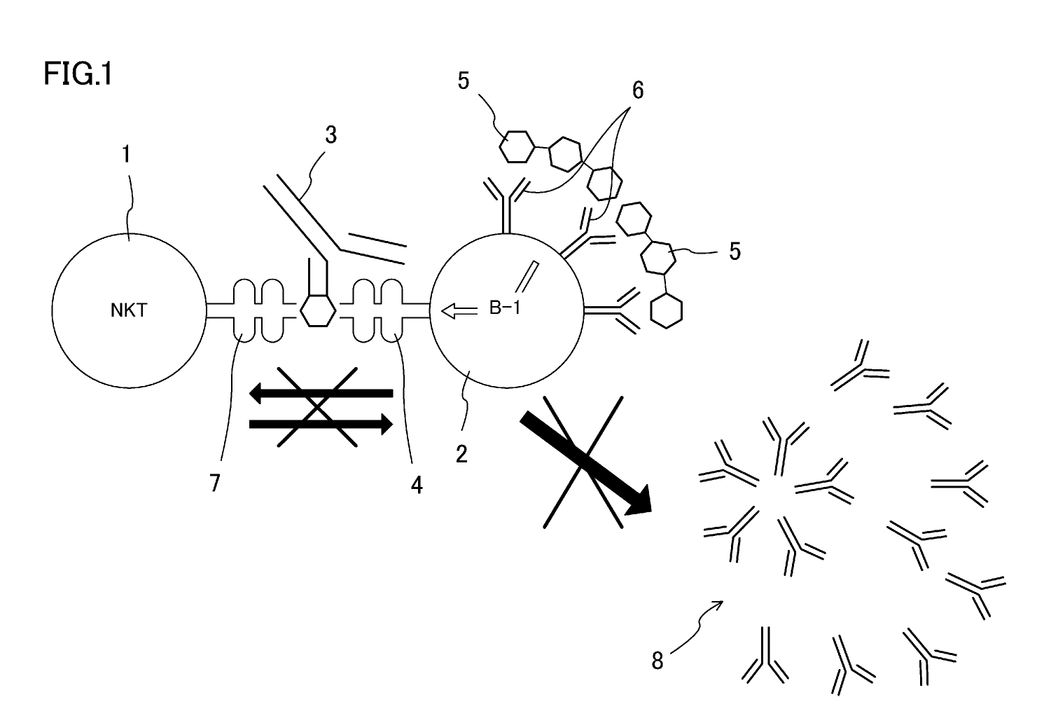

FIG. 1 schematically illustrates a mechanism of the process described above. In FIG. 1, reference character 1 denotes an NKT-cell, reference character 2 denotes a B-1 cell, and reference character 3 denotes an anti-CD1d antibody.

As shown in FIG. 1, the B-1 cell 2 has, at its surface, a CD1d molecule 4 and an antigen receptor 6 which reacts with a blood group carbohydrate chain antigen 5. The NKT-cell 1 has, at its surface, an antigen receptor (iTCR) 7 which reacts with the CD1d molecule 4 of the B-1 cell 2. Signal transduction between the NKT-cell 1 and the B-1 cell 2 is performed via the iTCR 7 and the CD1d molecule 4. The CD1d molecule 4 is not intrinsic in the B-1 cell 2, but also exists in a B-2 cell.

It is expected that when the blood group carbohydrate chain antigen 5 is bonded to the B-1 cell 2, this blood group carbohydrate chain antigen 5 is presented on the CD1d molecule 4, antigen information is transduced to the NKT-cell 1, and then an anti-blood group antibody 8 is produced by cooperation of the NKT-cell 1 and the B-1 cell 2.

If this expectation is true, impairment of signal transduction between the NKT-cell 1 and the B-1 cell 2 by the anti-CD1d antibody 3 bonded to the CD1d molecule 4 can inhibit production of the anti-blood group antibody 8 as shown in FIG. 1.

To verify this hypothesis, the inventors conducted the following experiment. Mice received anti-CD1d antibodies and mice received isotype-matched control antibodies (isotype control) were immunized with human group A red blood cells. Then, time-dependent productions of anti-A IgM antibodies and anti-A IgG antibodies were compared (where detailed description will be given in a third example).

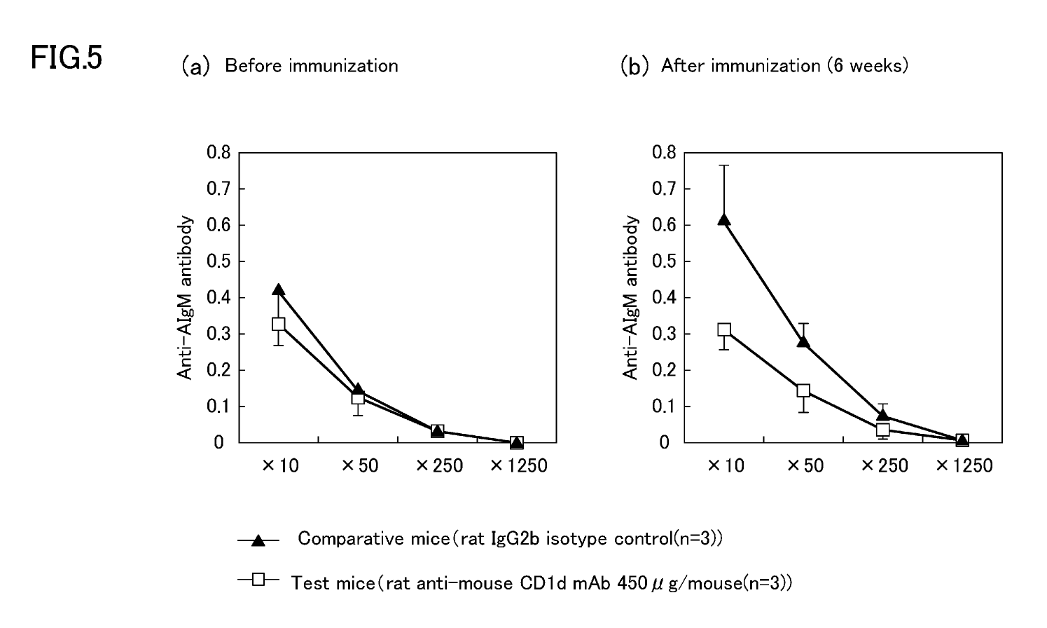

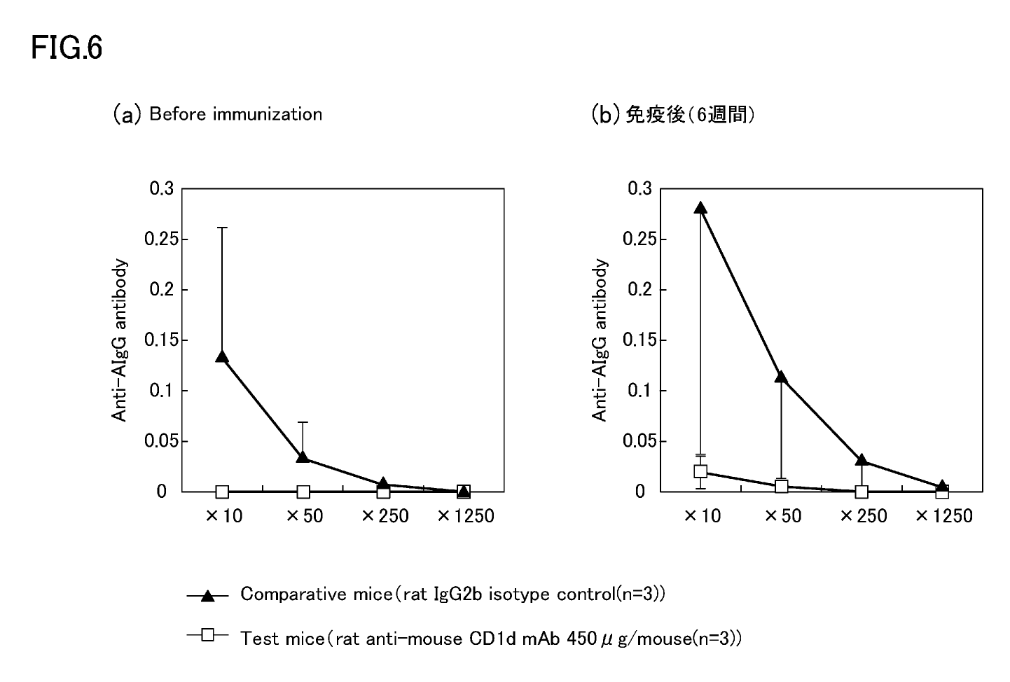

This comparison showed that normal antibody production was observed in mice received isotype control antibodies, and that inhibition of production of anti-blood group antibodies and inhibition of class switching of antibodies were observed in mice received anti-CD1d antibodies as in knockout mice (see FIGS. 5(a), 5(b), 6(a), and 6(b)).

Accordingly, if signal transduction between the NKT-cell and the B-cell is impaired using an antibody-mediated rejection inhibitor such as anti-CD1d antibodies to inhibit production of antibodies, it is possible to achieve specific inhibition of production of anti-blood group antibodies without impairing antibody production against general peptide antigens.

In the case of mice, since anti-CD1d antibodies are already commercially available in the market, an antibody-mediated rejection inhibitor for mice can be easily obtained by purchasing and appropriately adjusting the anti-CD1d antibodies.

If the antibody-mediated rejection inhibitor for mice administered to mice in such a manner that a dose of anti-CD1d antibodies was 400 to 600 μg/mouse, preferably 450 μg/mouse, specific inhibition of production of anti-blood group antibodies in these mice can be achieved, thereby enabling organ transplantation irrespective of the blood groups.

In the case of humans, it is sufficient to administer monoclonal human anti-CD1d antibodies using a series of known techniques for forming human monoclonal antibodies. Then, the monoclonal human anti-CD1d antibodies are mixed with preservatives and other supplemental drugs such as nutrients according to conventional methods, and adjusted as injections, thereby obtaining an antibody-mediated rejection inhibitor for humans.

The antibody-mediated rejection inhibitor is administered at a predetermined dose (which can be obtained by conversion based on the weight with the assumption that the weight of a mouse is 30 g). Then, specific inhibition of production of anti-blood group antibodies can be achieved, thereby enabling organ transplantation without impairing normal immunoreaction, irrespective of blood groups.

In this process, these anti-CD1d antibodies are effective in cells which have been already differentiated to B-1 cells. Thus, administration of the anti-CD1d antibodies will be more effective when combined with administration of a calcineurin inhibitor which inhibits differentiation of B-0 cells to B-1 cells.

The present disclosure might also be applicable to xenotransplantation, e.g., transplantation where pig organs which are closely analogous to human organs in terms of biology and anatomy are used instead of human organs.

Specifically, pig organs contain Galα1-3Galβ1-4GlcNAc (Gal) carbohydrate chains and N-glycolylneuraminic acid (NeuGc) carbohydrate chains, which are targets of natural antibodies to cause antibody-mediated rejection. Since these Gal carbohydrate chains and NeuGc carbohydrate chains are analogous to blood group carbohydrate chain antigens, the antibody-mediated rejection inhibitor according to the present disclosure is likely to be also effective in xenotransplantation between humans and pigs.

As described above, the antibody-mediated rejection inhibitor of the present disclosure can be relatively easily put into practical use, and the use of this antibody-mediated rejection inhibitor is expected to provide safe organ transplantation from ABO-incompatible donors. For this reason, the antibody-mediated rejection inhibitor of the present disclosure can solve the problem of donor shortage.

### First Example

To analyze the influence of NKT-cells on production of antibodies against blood group carbohydrate chain antigens, Balb/c CD1d⁻/⁻ mice (which lack CD1d as a ligand for iTCR and known to show a significant decrease in NKT-cells; knockout mice, n=4) and comparative Balb/c wild-type mice (n=5) were immunized with human group A red blood cells (8×10⁸/mouse), production of anti-blood group A antibodies were measured by ELISA.

The human blood group A red blood cells were immunized in the following manner. Blood is collected from blood group A healthy volunteers, twice washed in PBS, and diluted to 8×10⁸/ml. Then, 1 (one) ml of this diluted cells were administered in the abdominal cavities of mice, and twice immunized at a 1-week interval.

Detection of anti-blood group A antibody titers by ELISA was conducted in the following manner. Polystyrene 96-well flat-bottom plates (costar) were coated at 4° C. for 8 hours with 5 μg/ml of A-bovin serum albumin (BSA) (Dextra), or 5 μg/ml of BSA (Roche) as a background control. Then, the plates were washed, and blocked by 1 (one) % BSA. Diluted mouse serum was added to each well, and incubated at room temperature for 2 hours. Thereafter, 0.25 μg/ml of anti-mouse IgM- or IgG-HRP (Southern Biotech) was added as secondary antibodies, and incubated at room temperature for one hour. After washing the plates, 0.1 mg/ml of O-phenylendiamine (SIGMA) was added for color development, and absorbance at 492 nm was measured by a multi-plate reader (COLONA). The absorbance of the BSA background plates was subtracted from the absorbance of the A-BSA coated plates, and the obtained difference was used as anti-blood group A antibody titers (see NON-PATENT DOCUMENT 1).

FIGS. 2(a), 2(b), 3(a), and 3(b) show the results of the above detection. The graph of FIGS. 2(a) and 2(b) show changes in the amount of anti-A IgM antibodies. FIG. 2(a) shows values before immunization, and FIG. 2(b) shows values obtained two weeks after immunization. In each graph, the ordinate represents the absorbance indicating antibody amount, and the abscissa represents the dilution factor of added serum.

As shown in the graph of FIG. 2(a), no difference in the amount of anti-A IgM natural antibodies was observed between the knockout mice and the wild-type mice before immunization. On the other hand, as shown in the graph of FIG. 2(b), an increase in the amount of anti-A IgM antibodies was observed after immunization in the wild-type mice, whereas the amount of anti-A IgM antibodies hardly changed between before and after immunization in the knockout mice.

The graphs shown in FIGS. 3(a) and 3(b) show changes in the amount of anti-A IgG antibodies. FIG. 3(a) shows values before immunization, and FIG. 3(b) shows values obtained six weeks after immunization. In each graph, the ordinate represents the absorbance indicating antibody amount, and the abscissa represents the dilution factor of added serum, as in FIGS. 2(a) and 2(b).

As shown in the graph of FIG. 3(a), anti-A IgG antibodies were not observed in any of mice before immunization. On the other hand, as shown in the graph of FIG. 3(b), production of anti-A IgG antibodies was observed in the wild-type mice, whereas no production of anti-A IgG antibodies was observed in the knockout mice.

### Second Example

To analyze the influence of NKT-cells on production of antibodies against general peptide antigens, the knockout mice and Balb/c wild-type mice (MHC haplotype d) were immunized with thymocytes (20×10⁶/mouse) of C57BL/6 wild-type mice (MHC haplotype b), and the production amount of anti-allo-MHC antibodies was measured with a flow cytometer (n=6, untreated group for comparison: n=3).

Allo-MHC antigens were immunized in the following manner. Thymi of C57BL/6 wild-type mice were extirpated, and mashed with dishes. From the resultant thymi, red blood cells were removed with ACK lysing solution (155 mM NH4C1, 10 mM KHCO3, 1 mM EDTA-2Na, and PBS, pH 7.4), and thymocytes were isolated, and diluted to 20×10⁶/ml with a 199 medium. Then, 1 (one) ml of the diluted thymocytes were administered in the abdominal cavities of mice, and twice immunized at a 1-week interval.

Anti-allo-MHC antibodies (anti-MHC haplotype b antibodies) were detected in the following manner. Thymocytes of C57BL/6 wild-type mice were isolated in the manner described above, and diluted to 10×10⁶/ml with a flow cytometery (FCM) medium (PBS containing 0.1% BS and 0.1% sodium azide). Then, 10 μl of subject plasma was added to each 1×10⁶ cells, and incubation was performed at 4° C. for one hour. As secondary antibodies, 10 μl of anti-mouse IgM or IgG1, 2a/b, 3-biotin was added, and incubation was performed at 4° C. for 30 minutes. Further, streptavidin-PE was added, and incubation was performed at 4° C. for 15 minutes. The resultant cells were analyzed with FACSCalibur flow cytometer (Becton Dickinson), and the mean fluorescein intensity (MFI) was used as an antibody amount.

The results of the analysis are shown in FIGS. 4(a), 4(b), and 4(c). FIGS. 4(a), 4(b), and 4(c) show changes in the antibody amount of IgG for respective sub-classes, and FIG. 4(a) is associated with IgG1, FIG. 4(b) is associated with IgG2a/b, and FIG. 4(c) is associated with IgG3. In each graph, the ordinate represents the antibody amount with the mean fluorescein intensity (MFI), and the abscissa represents the elapsed period.

In the untreated group, no time-dependent change in the antibody amount was observed in any individual, but normal production of anti-allo-IgG type antibodies was observed in both of the knockout mice and the wild-type mice. No difference in class switching was observed between the sub-classes.

### Third Example

To analyze whether or not impairment of signal transduction between NKT-cells and B-1 cells by anti-CD1d antibodies can inhibit production of anti-blood group antibodies, 450 μg of anti-CD1d antibodies (rat anti-mouse CD1d monoclonal antibodies, clone: 1B1) per a mouse was administered in the abdominal cavities of Balb/c wild-type mice (test mice, n=3). In the same manner, 450 μg of rat IgG2b antibodies (isotype control) per a mouse was administered in the abdominal cavities of mice (comparative mice, n=3).

After 24 hours, these mice were twice immunized with human group A red blood cells at a 1-weel interval. Then, production of anti-blood group antibodies was measured by ELISA in the manner described above.

FIGS. 5(a), 5(b), 6(a), and 6(b) show the results of the measurement.

The graphs shown in FIGS. 5(a) and 5(b) show changes in the amount of anti-A

IgM antibodies. FIG. 5(a) shows values before immunization, and FIG. 5(b) shows values obtained six weeks after immunization. In each graph, the ordinate represents the absorbance indicating antibody amount, and the abscissa represents the dilution factor of added serum.

As shown in the graph of FIG. 5(a), no difference in the amount of anti-A IgM natural antibodies was observed between the test mice and the comparative mice before immunization. On the other hand, as shown in the graph of FIG. 5(b), an increase in the amount of anti-A IgM antibodies was observed after immunization in the comparative mice, whereas the amount of anti-A IgM antibodies did not change between before and after immunization in the test mice.

On the other hand, the graphs shown in FIGS. 6(a) and 6(b) show changes in the amount of anti-A IgG antibodies. FIG. 6(a) shows values before immunization, and FIG. 6(b) shows values obtained six weeks after immunization. In each graph, the ordinate represents the antibody amount, and the abscissa represents the dilution factor of added serum, as in FIGS. 5(a) and 5(b).

As shown in the graphs of FIGS. 6(a) and 6(b), anti-A IgG antibodies increased (i.e., were produced) after immunization and class switching was observed in the comparative mice, whereas anti-A IgG antibodies did not increase (i.e., were not produced) after immunization and no class switching was observed in the test mice.

### Fourth Example

To confirm that the same advantages as those in the case of mice can be obtained for humans, a test was conducted using human lymphocyte chimeric mice.

Human lymphocyte chimeric mice were obtained in the following manner. Severe combined immunodeficiency mice (NOD.Cg-Prkdc^{scid} Il2rg^{tm1Sug}/Jic; produced by Central Institute for Experimental Animals) was purchased. Then, 20×10⁶, per a mouse, of peripheral blood lymphocytes collected from blood group O healthy volunteers was administered in the abdominal cavities of the severe combined immunodeficiency mice.

Six days after lymphocyte administration, 500 μg of anti-CD1d antibodies (mouse anti-human CD1d monoclonal antibodies, clone: CD1d42) per a mouse was administered in the abdominal cavities (test mice, n=3). In the same manner, 500 μg of mouse IgG1 antibodies (isotype control clone: 107.3) per a mouse was administered in the abdominal cavities (comparative mice, n=4). Since this test is directed to humans, monoclonal antibodies specific to human CD1d molecules were produced, and used.

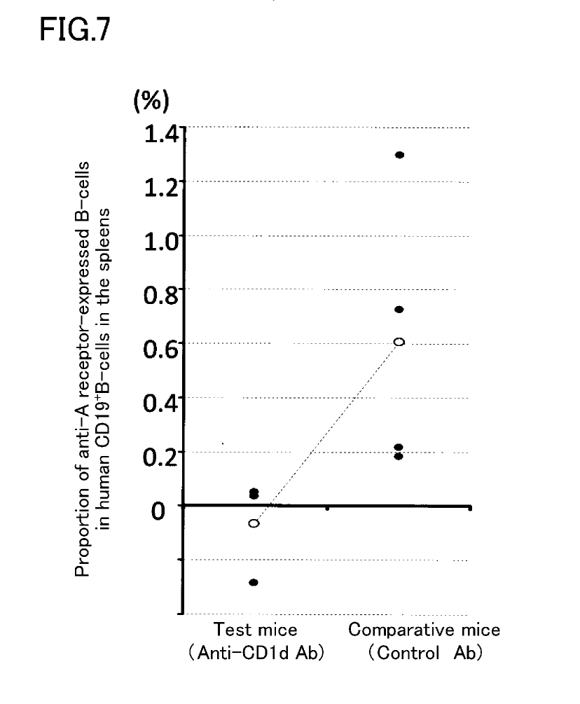

Seven days after lymphocyte administration, the mice were immunized with human group A red blood cells (where 8×10⁸ cells per a mouse). Then, 21 days after lymphocyte administration, spleens were collected from the mice, and the proportion of anti-A receptor-expressed B-cells in human CD19⁺B-cells in the spleens were measured by a flow cytometer. The results are shown in FIG. 7

FIG. 7 shows the proportion of anti-A receptor-expressed B-cells in human CD19⁺B-cells in the spleens of human lymphocyte chimeric mice. In this graph, black circles represent values of respective mice, and open circles represent the average of the values.

As shown in FIG. 7, human B-cells which recognize A-antigens in the spleens of the test mice administered with anti-CD1d antibodies show a proportion significantly lower than that in the spleens of the comparative mice administered with control antibodies. That is, anti-CD1d antibodies can also achieve specific and significant inhibition in the response against blood group antigens of human B-cells.

### INDUSTRIAL APPLICABILITY

The present disclosure is applicable to inhibition of development of autoimmune diseases as well as organ transplantation and xenotransplantation of, for example, mice and humans.

### DESCRIPTION OF REFERENCE CHARACTERS

1 NKT-cell
2 B-1 cell
3 anti-CD1d antibody
4 CD1d molecule
5 carbohydrate chain antigen
6 antigen receptor
7 iTCR
8 antibody

1. An antibody-mediated rejection inhibitor for impairing signal transduction between NKT-cells and B-cells to inhibit production of antibodies, the inhibitor comprising anti-CD1d antibodies.

2. The antibody-mediated rejection inhibitor of claim 1, wherein the B-cells include B-1 cells.

3. The antibody-mediated rejection inhibitor of claim 1, wherein the antibodies to be a target for producing inhibition include antibodies against carbohydrate chain antigens.

4. The antibody-mediated rejection inhibitor of claim 3, wherein the carbohydrate chain antigens include blood group carbohydrate chain antigens.

5. The antibody-mediated rejection inhibitor of claim 4, wherein the antibodies to be a target for producing inhibition do not include antibodies against peptide antigens.

6. The antibody-mediated rejection inhibitor of claim 1, wherein the anti-CD1d antibodies are monoclonal antibodies.

7. The antibody-mediated rejection inhibitor of claim 6, wherein the anti-CD1d antibodies are human monoclonal antibodies.

8. A method for screening of an antibody-mediated rejection inhibitor, the method comprising the steps of:
administering a target component in abdominal cavities of mice to obtain test mice, while administering an antibody component to be inhibited in abdominal cavities of other mice to obtain comparative mice; and
immunizing each of the test mice and the comparative mice with red blood cells, thereby measuring production of anti-blood group antibodies.
